(19) United States
(12) Patent Application Publication
(10) Pub. No.: US 2007/0083092 A1
(43) Pub. Date: Apr. 12, 2007

(54) External exercise monitor

(76) Inventors: Anthony J. Rippo, Burton, WA (US); Bruce E. Jackson, Vashon, WA (US)

Correspondence Address: Bruce E. Jackson, P.O. Box 836, Vashon, WA, 98070, US

(21) Appl. No.: 11/594,287

(22) Filed: Nov. 9, 2006

Related U.S. Application Data
(63) Continuation-in-part of application No. 11/245,381, filed on Oct. 7, 2005.

(51) Int. Cl. A61B 5/00

(52) U.S. Cl. 600/307

(57) ABSTRACT

A device to determine internal physiological parameters based upon the measurement of phenomena external to a person's skin. In one embodiment, a wristwatch apparatus is provided with sensors capable of measuring, for example, transpired vapors from the wearer's skin, sweat produced through the skin, or saliva deposited on the device. The measurement of predetermined parameters in these external media is compared against a library of internally measured parameters to determine the level of stress the wearer is experiencing.

### CLAIM TO DOMESTIC PRIORITY

This is a Continuation-in-Part application claiming priority to prior U.S. patent application Ser. No. 11/245,381, filed Oct. 7, 2005.

### STATEMENT REGARDING FEDERALLY-SPONSORED R&D

Not relevant.

### FIELD OF THE INVENTION

The present invention, in its most general embodiment, relates to a device removably attached to the surface of a person's skin for monitoring external indicia that correlate with internal physiological parameters that are the result of exercise.

### BACKGROUND OF THE INVENTION

It has long been a goal of exercise physiologists and coaches to know how the bodies of athletes or non-athletes react to different levels of exercise. There are many internal physiological indicia of different levels of exercise, whether it be moderate exercise (recreational walking) or extreme (competitive marathon runners), and whether the person being measured is an ordinary non-athletic citizen or an Olympic athlete.

Most exercise monitors are designed for use in, for example, an indoor facility or gym, where one goes to exercise for the purpose of fitness. Monitors used in these environments are typically for the purpose of determining the number of calories expended, the impact on cardiovascular fitness, and the like. Such devices either store the information internally or transmit the information to a central repository. It is well known that a couple of internal physiological parameters, such as heart rate or pulse, can be measured externally, and indeed are done routinely in many environments world-wide. Additionally, devices have been proposed to measure pressure-sensitive physiological data, such as the measurement of heart rate or pulse rate.

Such devices have been devised to measure one or more attributes of a person undergoing exercise. For example, U.S. Pat. No. 6,358,187 discloses a device that provides an exercising person information at any desired period of time about his pulse rate at that instant. The information is given as vocal information, and the device comprises an ECG unit with electrodes adapted to be attached to the human body, one of which forms part of an earphone, filter and amplification means, a speech synthesizer, and means for evaluating instantly the ECG and for giving a vocal indication of the instant pulse rate. U.S. Pat. No. 6,269,314 discloses a blood sugar measuring device to measure blood sugar either non-invasively or with only slight invasiveness which is capable of better measurement accuracy. The device receives as input measured data related to blood sugar level such as patient mealtime and how much food he had, or if he had an insulin injection and adjusts the measured blood sugar value based on the data which are previously input based on the above measured data. U.S. Pat. No. 6,251,048 discloses an electronic activity monitor for monitoring exercise that comprises an activity detector responsive to motion associated with the performance of the activity to output a corresponding signal.

Pressure sensitive devices such as those disclosed in U.S. Pat. Nos. 6,126,572 and 6,823,036 provide data regarding heart rate measured by pressure devices, and ECG monitoring devices using electrodes attached to the skin, as disclosed in U.S. Pat. No. 5,314,389, are exemplary of state-of-the-art exercise monitors.

Similarly, U.S. Pat. No. 5,516,334 comprises an interactive exercise monitor that computes and displays time, distance, pace, and energy expended by a user performing a repetitive workout around a predetermined course, using a stationary transmitter located along the workout course and a receiver carried by the user. The stationary transmitter emits a limited range signal that is detected by the receiver each time the user passes the transmitter during the workout. U.S. Pat. No. 5,314,389 discloses a device that provides an exercising person information at any desired period of time about his pulse rate at that instant using an ECG unit with electrodes adapted to be attached to the human body, one of which forms part of an earphone, filter and amplification means, a speech synthesizer, and means for evaluating instantly the ECG and for giving a vocal indication of the instant pulse rate. The device is of special value for the monitoring of the pulse rate during jogging and similar types of exercise, the information being provided vocally.

It would be of great benefit to ordinary citizens on the one hand, and to athletes, coaches and trainers on the other hand, to be able to determine, in real time, the internal physiological impact of a particular exercise regime. By way of example only, and without intending to be limiting, rehabilitative physicians and athletic coaches/trainers would find value in knowing the levels of internal hydration, electrolytic balance, lactic acid concentration, glucose levels, catecholamine levels, c-reactive protein, and other measurable internal physiological attributes.

For example, if during the running of a marathon, a coach or athlete could determine real time levels of lactic acid, an undue increase of lactic acid could be counteracted by taking a measured amount of a buffering solution during the race. If a patient after undergoing heart surgery could easily determine during a walk to the office that her level of electrolytes (such as sodium or potassium) were too high, she could take sufficient water to safely bring the electrolyte concentration into proper balance.

Because the value of the device as proposed lies in its ease of use and ready portability, it must be easily attached to the exterior of the body and must be non-invasive, or at least not actively invasive. Because it is not intended to be a permanent monitoring device, it must be portable and removable when not needed. With miniaturization, a number of monitors for different physiological parameters are advantageously packaged into a single device. Preferably, the device is capable of measuring at least some of the physiological parameters so that it can be used either by swimmers or in inclement weather. And finally, the device should be capable of itself displaying the results of its monitoring, and either storing measurements on-board or sending measurements to a remote receiver for storage.

### SUMMARY OF THE INVENTION

In its broadest embodiment, the present invention comprises a device removably attached to the surface of an exercising person's skin for monitoring internal physiological parameters of such person, and comprises at least one sensor capable of measuring an external phenomena that is a direct result of that person's internal physiological response to exercise.

In another embodiment of the invention, the device specifically measures external attributes that may be correlated with such internal physiological parameters as hydration, electrolytic balance, lactic acid concentration, catacholamine concentration, glucose levels, c-reactive protein levels, and the like. While the device does not measure these parameters directly, it uses measured external attributes and compares such measurements to stored libraries of correlations. Such libraries may be developed and stored for an individual, or may be collected and stored for a defined population.

In yet another embodiment, the present invention comprises a method of monitoring at least one internal physiological parameter of an exercising person undergoing exercise, comprising the steps of removably attaching a monitoring device to the surface of the person's skin, providing at least one sensor in the monitor to detect external phenomena adjacent to the person's skin, correlating the external phenomena to a measure of an internal physiological response to exercise, and displaying the internal physiological response.

Therefore, there is a need for a removable, external device having sensors collecting real-time information that can be correlated to internal physiological parameters that are responses to exercise.

### BRIEF SUMMARY OF THE DRAWINGS

Not relevant

### DETAILED DESCRIPTION OF THE INVENTION

There are a number of situations in which individuals may wish to understand the extent of their internal physiological status as affected by, and in response to, exercise. As used herein, the word “exercise” is to be defined as broadly as possible and comprises virtually any activity more rigorous than experienced when a body is at rest, and may be as limited as isometric exercises while sitting or supine, normal walking, or may be as rigorous as running a competitive marathon. While the physiological response to this exercise can be monitored by any number of standard, and well known, means that involve invasive techniques (such as the drawing and analysis of blood or other fluids, implantation of electrodes, indwelling catheters containing sensors), or by sophisticated analytical tools typically found only in hospitals or state-of-the-art out-patient facilities, such methods of tracking internal physiological responses to exercise is prohibitively expensive and difficult or impossible to monitor in real time. Some physiological responses to exercise may be monitored while exercising (for example, on a treadmill in a clinic environment), currently the ability to monitor internal physiological responses to such exercises is quite limited. For example, heart rate and pulse rate may be monitored at pressure points on the surface of the skin, and ECG readings may be monitored by attaching electrodes to the skin, there is little ability to non-invasively measure real-time internal metabolic or physiological responses during any level of exercise. Therefore, it is to be understood that while there are a number of reasons to measure such physiological responses in a broad array of persons undergoing exercise, for ease of description, the description herein will focus on just one class of subjects, namely athletes. It is submitted that virtually anything described herein relating to athletes is equally applicable to the entire universe of individuals for whom it may be useful to non-invasively monitor real-time internal metabolic or physiological responses to exercise for whatever the reason.

The purpose of measuring the internal physiological responses to exercise is to determine whether or not during exercise the individual needs to adjust the exercise routine, or possibly ingest a supplement (possibly as simple as water to increase hydration, or as complex as an electrolytic solution to modify an electrolyte imbalance) to modify an undesirable physiological response to exercise. Likewise, the device of the present invention may be utilized to indicate whether an athlete is overtraining or expending an inordinate amount of energy during a competition, and can adjust the exercise routine accordingly. For example, recent deaths of football players have been at least preliminarily attributed to electrolytic imbalances or heart rhythm irregularities, which if observed in real time, may be able to save lives if the exercise is terminated and the imbalance corrected. Likewise, if hydration levels in contestants in marathons or triathlons are observed, steps may be taken to reverse adverse physiological conditions and enable the contestant to improve his or her end result.

In its broadest embodiment, the present invention comprises a device removably attached to the surface of a person's skin that is capable of non-invasively monitoring real-time internal metabolic or physiological responses of said person while undergoing exercise, comprising at least one sensor in said device capable of measuring external phenomena adjacent to the person's skin that are a direct result of internal physiological response to exercise. As used herein, “removably attached” is understood to mean a non-invasive apparatus that is completely external to the individual's skin, such as a wristwatch-type device, or other device provided with sensors adapted to measure external phenomena that can be correlated to internal physiological responses to exercise. Further, as used herein, “adjacent to a person's skin” is intended to mean not measurements of the skin itself, or by contact against the person's skin, but rather of a phenomena that is external to the person's skin, such as (without limitation) the measurement of transpired water vapor, sweat, or saliva.

There are a wide variety of internal physiological parameters that one may wish to measure. Without intending to be limited in any way, such parameters may comprise hydration, electrolytic balance, lactic acid concentration, glucose concentration, estrogen or other hormone levels, catecholamine levels, cortisone level, c-reactive protein levels, IGA, IL-6, biomarkers of oxidative stress, and other similar parameters. It is to be understood that the present invention is limited only to those internal physiological parameters that may change as a result of exercise, and that may be correlated to a measurable or monitored external phenomena that is the result of such exercise. Additionally, the external phenomena may be measured by any non-invasive means available for sensors to monitor, such as respiration through the lungs, transpiration through the skin, saliva, sweat, urine, or the like.

The sensors utilized herein may be any single sensor or combination of sensors that measure one or more of these external phenomena. A number of such sensors may be described herein, and it is to be understood that the invention described and claimed herein is not limited solely to those described, but can be practiced by any sensor available that is capable of measuring an external phenomena that is capable of measurement and correlation to an internal metabolic or physiological response to exercise. The device manufactured according to the instant invention may have a single sensor, or a plurality of sensors, therein.

In a preferred embodiment, for any internal metabolic or physiological parameter one desires to measure, a library of (1) measurements of external phenomena measured in a “resting” exercise state is obtained, and then (2) external phenomena at a wide range of exercise states are measured, which can then be correlated against a similar library of internal parameters to determine whether the internal physiological parameter is within a “normal” range or is out-of-norm. Software must be provided that instantaneously compares the measured external phenomena with the corresponding resting exercise state so as to provide an absolute number representing the physiological parameter. The absolute number representing the internal metabolic or physiological parameter is then used, for any specific athlete, to determine the individual's physiological response to the exercise level being experienced. An appropriate response to the absolute number is then determined, and an indication is provided whether the measurement is within a normally expected range for the measurement.

For example, a library of “typical” internal metabolic or physiological responses to a particular exercise level over a representative population of similarly-situated individuals, or individual-specific responses to particular exercise level, may be developed and stored in the device. When the external phenomena is measured during exercise, the correlation between that being measured in real time may be compared against the library, and the individual's specific response may be determined. As just one example, a marathon runner may check her hydration level as measured in relative humidity immediately adjacent her skin at various points during a 26 mile race, and the results may indicate when additional fluids need to be taken. Or, specific electrolytes may be measured in sweat or saliva external to the skin and if a deficiency in an internal metabolic or physiological parameter is detected, fluids containing the deficient electrolytes may be administered.

The following Table 1 is an exemplary listing of internal physiological parameters that may be of interest to one undergoing exercise, with a normal range of the parameter provided, as well as an indication of where a dysfunction may arise, the cause (or “alarm”) of the dysfunction, and a possible remediation for the dysfunction. Numerous other parameters may be of interest to exercise physiologists or physicians and the list of Table 1 is by no means exhaustive—the list of possible parameters is limited solely by the capability to design and build sensors to detect external of or adjacent to a person's skin.

TABLE 1

| Internal | | | | |
|---|---|---|---|---|
| Physiological | | | | |
| Parameter | Normal | Dysfunction | Alarm | Remediation |
| Hydration | 0.73 | <0.73 | dehydration | hydrate |
| (ratio of water: fat | | >0.73 | overhydration | drink |
| free body mass) | | | | electrolytes |
| Electrolyte | (mEq/L) | | | |
| Sodium | 135-146 | >upper range | dehydration | hydrate |
| | | limit | | |
| Potassium | 3.5-5.5 | | | |
| Chloride | 95-112 | | | |
| CO2 | 8.5-10.3 | | | |
| Calcium | ?? | | | |
| Phosphorus | ?? | | | |
| Lactic Acid | 4.5-19.8 | >upper range | overexertion | Slow pace |
| | mg/dl | limit | | |
| Catacholamine | 200-1100 | <lower range | overtraining/ | Rest or slow |
| (norephinephrine) | mg/L | limit | overexertion | pace |
| Cortisone | 6-23 mg/dl | <lower range | overtraining | Rest, sweat |
| | | limit | | |
| C-reactive | <0.6 mg/dl | >amount | overtraining/ | Rest, evaluate |
| Protein | | | inflamation | |
| Glucose | 80-100 | <lower range | hypoglycemia | Hydrate with |
| | mg/L | limit | | sugar water |
| EKG | Normal | Abnormal P, | Dehydration or | Proper fluids, |
| | electrical | prs, t waves & | overhydration | Evaluate! |
| | complex | intervals | | |

It should be obvious that the device of the present invention may be provided in any number of embodiments that permit the measurement of external phenomena in a manner that produces information necessary to monitor internal metabolic or physiologic responses to exercise. In a wristwatch embodiment, probes may be provided that are placed adjacent the skin so that they contact sweat and make appropriate measurements. Further by way of example only, a “dead space” may be provided beneath the wristwatch housing or casing in a manner that isolates it from external factors so that a humidity sensor may make reasonably accurate measurements of water vapor transpiring from the surface of the skin.

In the wristwatch embodiment, a housing will contain one or more sensors that are provided to make measurements of phenomena external to the person's body, in this case the wrist or hand. External media are analyzed as a means of calculating the level of stress in certain internal physiological parameters. As an example, a microsensor for humidity may be provided in a dead space external of the wearer's skin in the form of an ion selective electrode or an ion selective field effect transistor with a hydrophilic membrane. Regardless of the sensor used, it will have the capability to measure either the amount of water in the vapor trapped in the dead space as a measure of internal hydration, or alternatively the concentration of chemical analytes contained in such vapor.

In another embodiment of the present invention, sensors may be provided to contact liquid sweat of the individual and determine the levels of certain chemical analytes contained in the liquid sweat. For example in order to measure electrolyte balance, an ion selective electrode or ion selective field effect transistor may be used.

There are numerous internal physiological parameters that may be measured through analysis of saliva. The apparatus of the present invention, during a period of exercise, may be provided with means that can be licked by the individual, with the saliva being deposited directly upon or conveyed to sensors that measure the parameter of interest in the liquid saliva.

In any of the previous embodiments, the device may be advantageously provided with the capability to purge remnants of the external phenomena and any particular deposition of liquids or precipitates, and with the capability to additionally thereafter cleanse the sensors. Such purging and cleansing will prevent distorted measurements caused by a build-up on the sensors, and thereby provide more accurate measurements over time.

Those skilled in the art will immediately appreciate the numerous configurations and embodiments of sensors that may be utilized in making the measurements required by the invention herein. It will be immediately appreciated by those of skill in the art that the invention herein does not lie in the particular sensors that may be used, but rather the invention resides in a device capable of non-invasively measuring in real time one or more phenomena external to the wearer's skin surface that result from internal metabolic or physiological responses to exercise.

The electronic components of the invention will preferably be custom fitted to a small, portable wearable device as described herein. After wiring the device in a manner to permit capture of the information set forth above, the information may be either stored in the wearable device or transmitted to a remote device. In either case, the readings of external phenomena from the sensors will provide real-time information regarding the parameter of interest, which will be compared against the library for determination of the status of the internal physiological parameter. In most cases, the readings will be displayed in a form meaningful to the person or medical personnel. If the status of the internal physiological parameter falls outside a predetermined “normal” state, a warning in the form of audible signal, vibration, visible light signal, or the like may be automatically activated to warn of an out-of-norm event that needs corrective action.

External sensor measurements may be taken periodically and the results stored. Software may be provided to evaluate trends in external phenomena to indicate an imminent out-of-norm event, thereby enabling the individual to take corrective action prior to experiencing the event.

It should be appreciated that devices known in the prior art may be incorporated into the device of the present invention. For example, pulse monitors and ECG monitors may be incorporated with the other monitors described herein.

In a further embodiment of the invention, which may be particularly (but not exclusively) applicable to high-performance athletes, it is useful to determine an individual's state of hydration. An historical library of an individual's resting electrocardiogram (EKG) is produced. Then, the same sort of library is produced for that individual when engaged in various levels of exercise, presumably producing a different EKG pattern. The exercise-induced EKG library is also provided with examples wherein the individual is in a state of normal hydration, under (de)hydration, and over hydration. Having baseline EKG libraries to refer to enables either a physician or the device itself to determine within which state of hydration an athlete is performing during exercise when the individual's EKG is obtained during the period of exercise. A software program, readily familiar to those of ordinary skill in this art, may be written to enable the real-time comparison of an athlete's EKG against the stored library to determine a real-time state of hydration.

It is well known that potassium is the primary intercellular electrolyte, and that serum potassium level is a relatively accurate predictor of hydration levels. As relative serum potassium levels rise (hyperkalaemia), one's EKG changes dramatically. There may be multiple reasons one's serum potassium levels can rise, but in this case the causation may be from dehydration common to one undergoing a rigorous exercise regimen. Potassium levels above about 5.5 mEq/l may produce an “abnormal” EKG, or at least an EKG different from an EKG measured with proper potassium levels. The effect of hyperkalaemia on the cell membrane is to decrease the resting membrane potential, and decrease the duration of the action potential and refractory period, which are potentially arrhythmogenic. The classic EKG functions that change during hyperkalaemia include (1) tall peaked T-waves, (2) reduction in amplitude and eventually loss of the P-wave, and (3) bizarre widening of the QRS interval. These EKG changes can be explained by the electrolytes physiological effect on myocardial cells. Mild levels of hyperkalaemia are associated with acceleration of terminal repolarisation, relulting in T-wave changes. The most common changes seen in the T-waves are “tenting” or “peaking”, and are considered to be the earliest abnormalities seen in the EKG. Mild to moderate hyperkalaemia causes depression of conduction between adjacent cardiac myocytes, resulting in prolongation of the PR and QRS intervals as potassium levels rise. P-wave amplitude disappears early because of the sensitivity of atrial myocytes to hyperkalaemia. Other wave functions that change with abnormally high potassium levels may be correlated to resting functions and may result in mild to severe EKG abnormalities.

Any of the above abnormalities identified in EKG functions correlated to high potassium levels resulting from dehydration may be monitored and utilized to predict dehydration in an individual. By monitoring these EKG functions, one may be able to predict a relative level of hydration in order to maintain optimal levels of hydration. While the invention has been described herein in relation to elevated potassium levels as a predictor of dehydration (as evidenced through an EKG), it is believed that other electrolytes or physical parameters may also be viewed (through changes in “normal” EKG readings) as predictors of relative dehydration level.

It is believed that, prior to actual dehydration occurring, the software program within the device may be utilized to identify trends in changing potassium levels or EKG functions, and predictions made so that adequate hydration may occur prior to loss of performance due to dehydration. For example, a process similar to that disclosed in U.S. Pat. No. 4,937,763 may be utilized to make such predictions and maintain performance at the highest possible level. In this manner, as one exercises and experiences a loss of hydration through sweating or internal metabolic processes, the software can be programmed to compile sequential EKG readings over time and predict a relative preset dehydration level prior to a preset dehydration level (evidencing onset of dehydration) actually being experienced. The software may notify the individual (or another person, such as a trainer, medical personnel, or coach) that while the individual is not currently dehydrated to a point of decreasing physical performance, water ingestion at a particular point will forestall such performance decline.

While real-time direct measurements of potassium are perhaps the clearest indication of hydration level, it involves invasive procedures not well adapted to an exercising person. Therefore, fluctuating (lowered) potassium levels resulting from dehydration, leading to the changes in EKG noted above (measured externally) will offer a relatively precise indication of relative hydration level.

EKG measurements may be made in a number of ways, the two most likely being direct measurement through a wrist-watch embodiment as described above, or by wireless transmission from conventional leads affixed to the chest wall to a remote receiver, either on the individual's body (e.g. in the wrist watch embodiment) or elsewhere.

It will be readily apparent to those of skill in this art that while the embodiment described immediately above is presented in the context of a high-performance athlete, those subject to either hypokalaemia or hyperkalaemia because of incipient or long term illness, may find the measurement of EKG, as a predictor of potassium levels, of medicinal benefit.

While preferred embodiments of the present invention have been shown and described, it will be apparent to those skilled in the art that many changes and modifications may be made without departing from the invention in its broader aspects. It is not the intent of applicant to limit the scope of the invention to any embodiment(s) disclosed herein, but rather the scope of the invention should be limited solely by the scope of the claims herein. The appended claims are therefore intended to cover all such changes and modifications as fall within the true spirit and scope of the invention.

What is claimed is:

1. A device removably attached to the surface of a person's skin for non-invasively monitoring internal physiological parameters of said person while undergoing exercise, comprising at least one sensor in said device capable of measuring external phenomena adjacent to the person's skin that are a direct result of internal physiological response to exercise.

2. The device of claim 1, wherein said sensor is adapted to measure water vapor transpired from said person's skin as an indication of relative humidity adjacent to said person's skin.

3. The device of claim 1, wherein a dead space is provided between the device and the person's skin to enable the measurement of relative humidity therein.

4. The device of claim 3, wherein said device correlates said measurement of water vapor with a library of measurements to predict a level of hydration within said person.

5. The device of claim 1, wherein said device is provided with a sensor adapted to analyze sweat exuded from said person's skin.

6. The device of claim 5, wherein said device correlates said analysis of sweat with a library of measurements to predict a level of a predetermined internal physiological parameter as a result of said exercise.

7. The device of claim 5, wherein said device is adapted to purge sweat and precipitates from the sensor and thereafter cleanse said sensor.

8. The device of claim 1, wherein said device is provided with a sensor adapted to analyze saliva deposited on the device by said person.

9. The device of claim 8, wherein said device correlates said analysis of saliva with a library of measurements to predict a level of a predetermined internal physiological parameter as a result of exercise.

10. The device of claim 9, wherein said device is adapted to purge saliva and precipitates from the sensor and thereafter cleanse said sensor.

11. The device of claim 1, wherein the device correlates the measurement of external phenomena with internal physiological parameters that may change as a result of exercise.

12. The device of claim 1, wherein the device comprises a monitor to record the person's electrocardiogram (EKG).

13. The device of claim 12, wherein the EKG is measured directly from the person's skin by the device.

14. The device of claim 12, wherein the EKG is measured from leads implanted on the person's chest wall and transmitted to a remote receiver.

15. The device of claim 12, wherein an exercise-induced EKG is compared in real time against a stored library of EKG readings.

16. The device of claim 15, wherein the stored library of EKG readings comprises EKG readings of the person during rest, during exercise with normal hydration, during exercise with low (de)hydration and during exercise with over hydration.

17. The device of claim 16, wherein if a comparison of real-time EKG readings is sufficient different from the library of stored EKG readings, an alert is issued of such difference.

18. A method of monitoring a person's internal physiological parameters that may change as a result of exercise, said parameters correlated to a measurable external phenomena that is the result of exercise, comprising
a. removably attaching to the surface of said person's skin a non-invasive device containing at least one sensor;
b. said at least one sensor adapted to measure external phenomena adjacent to the person's skin;
c. correlating said measurement to a library of external phenomena representing measurements under exercise;
d. further correlating said measurement with a library of internal phenomena representing internal physiological parameters; and
e. indicating whether said measurement is within a normally expected range for such measurement.

19. The method of claim 18, further comprising the step of providing said sensor with the capability to purge remnants of said external phenomena and to thereafter cleanse said sensor.

20. A device for monitoring the level of internal hydration of a person undergoing exercise, comprising:
a. an external monitor removably attachable to the surface of said person's skin, and
b. said monitor capable of measuring the level of internal hydration of said person by measuring physiological characteristics external to said person's skin.

21. The device of claim 20, wherein said external monitor is provided in the form of a wristwatch.

22. The device of claim 21, wherein said external monitor defines a dead space between said monitor and the person's skin, and further comprises a sensor capable of measuring levels of relative humidity in said dead space.

23. The device of claim 20, wherein said monitor comprises a processor to convert a level of relative humidity external to the person's skin to a level of internal hydration of said person.

24. The device of claim 23, wherein the level of internal hydration is displayed.

25. The device of claim 23, wherein if the level of internal hydration falls outside a predetermined level, the level of internal hydration a warning is provided to said person.

26. A method of monitoring at least one internal physiological parameter of a person undergoing exercise, comprising the steps of:
a. removably attaching a monitoring device to the surface of said person's skin;
b. providing at least one sensor in said monitor to detect external phenomena adjacent to said person's skin;
c. providing a library of internal physiological responses to a resting state and to exercise;
d. correlating said external phenomena to said library of internal physiological responses so as to provide an absolute measure of an internal physiological response to exercise, and
e. displaying said internal physiological response.

27. The method of claim 26, further comprising the step of detecting external airborne phenomena.

28. The method of claim 26, further comprising the step of detecting external phenomena in the form of liquid sweat.

29. The method of claim 26, further comprising the step of detecting external phenomena in the form of liquid saliva.

30. A device to enable the non-invasive monitoring of an individual's real-time level of hydration, comprising:
a. an apparatus adapted to record said individual's EKG in real time,
b. a software program to compare
(i) the real-time EKG
(ii) against a library of stored EKG readings representing the individual's EKG when normally hydrated, when under (de)hydrated, and when over hydrated, and
c. a display to publish a relative hydration level of the individual in real time as a correlation to the individual's EKG.

31. The device of claim 30, wherein EKG functions measured include T-waves, P-waves, PRS interval, and PR interval.

32. The device of claim 30 wherein said software program is adapted to compile sequential EKG readings over time and predict a relative preset dehydration level prior to said preset dehydration level actually being experienced by said individual.
